(19) United States
(12) Patent Application Publication
(10) Pub. No.: US 2005/0042772 A1
(43) Pub. Date: Feb. 24, 2005

(54) Removal of proteins from a sample

(75) Inventors: Stephen Naylor, Framingham, MA (US); Jiri Ademec, West Lafayette, IN (US); Michael Meys, Framingham, MA (US)

Correspondence Address: BOWDITCH & DEWEY, LLP, 161 WORCESTER ROAD, P.O. BOX 9320, FRAMINGHAM, MA, 01701-9320, US

(73) Assignee: Beyond Genomics, Waltham, MA (US)

(21) Appl. No.: 10/773,442

(22) Filed: Feb. 6, 2004

Related U.S. Application Data
(60) Provisional application No. 60/445,509, filed on Feb. 7, 2003.

(51) Int. Cl. G01N 33/543

(52) U.S. Cl. 436/518

(57) ABSTRACT

In various aspects, provided are methods and devices for the depletion of two or more proteins from a sample, such as, for example, blood serum, blood plasma, cerebrospinal fluid and/or urine samples. In various embodiments, the methods deplete three or more proteins from a sample by contacting the sample with at least one chromatographic medium, the at least one chromatographic medium being capable of removing albumin, IgG, and a third abundant protein from the sample. In various embodiments, the devices comprise a first chromatography column functionalized to substantially remove a first abundant protein from the sample; a second chromatography column in serial fluidic communication with the first chromatography column and functionalized to substantially remove a second abundant protein from the sample; and a first chromatography disk in serial fluidic communication with the second chromatography column and functionalized to substantially remove a third protein from the sample.

### CROSS REFERENCES TO RELATED APPLICATIONS

The present application claims the benefit of U.S. Provisional Application No. 60/445,509, filed Feb. 7, 2003, the entire contents of which are incorporated herein by reference.

### BACKGROUND OF THE INVENTION

Human plasma plays a key role in clinical medicine and drug discovery research as an indicator of abnormal metabolic processes because of its easy accessibility and high complexity. Plasma analysis is primary focused on the identification of enzymatic activities and the concentration of certain proteins generally known as protein biological markers as well as qualitative and quantitative determination of low molecular weight metabolites. Although the number of protein biological markers used currently is large, there are ongoing efforts being made in order to identify new biological markers or to increase sensitivity of current assays. This is often complicated by the presence of proteins present in high abundance in plasma, creating an inability to detect other potential markers or to increase the sensitivity of enzymatic assays.

### FIELD OF THE INVENTION

The present invention relates to removing proteins from a sample. More specifically, the invention involves depleting abundant proteins from blood plasma or serum.

### SUMMARY OF THE INVENTION

A major obstacle to proteomic analysis of complex samples such as blood plasma and serum is the presence of highly abundant proteins. For example, the presence of higher abundance proteins (for example, those present at greater than 1 mg/mL in serum) can interfere with the identification and quantification of lower abundance proteins (for example, those present at less than 1 ng/mL in serum). Further, for example, in human serum the concentration of albumin (typically 35-50 mg/mL) is nine, ten or more orders or magnitude higher than the immune system signal interlukin-6, which is normally present at levels of less than 5 picograms/mL. For example, with respect to samples such as blood serum, blood plasma, cerebrospinal fluid and urine, the top ten proteins by concentration in a sample are typically considered abundant proteins. Further, for example, the order by concentration of the top twenty proteins in human blood plasma, from highest to lowest, can be considered to be as follows (where hemoglobin has been excluded): albumin, IgG, transferrin, fibrinogen, IgA, alpha-2-macroglobulin, IgM, alph-1-antitrypsin, C3 complement, haptogloblin, apolipoProtein A-1, apolipoprotein B, alpha-1-acid glycoprotein, lipoprotein (a), factor H, ceruloplasmin, C4 complement, complement factor B, prealbumin, and C9 complement. However, as actual concentrations can vary in any one plasma sample the exact order of these abundant proteins can vary.

In general, proteins which are present at levels, for example, but without limitation, ten orders of magnitude higher than other lower concentration proteins can significantly interfere with the identification and quantification of the lower concentrations proteins. The removal of abundant proteins can facilitate the detection of lower concentration proteins by altering the dynamic range of the sample.

Accordingly, methods and devices according to the invention facilitate the removal of abundant proteins from sample such as, but without limitation, blood serum, blood plasma, cerebrospinal fluid, or urine. The objective of removing abundant proteins is to facilitate accessing and analyzing the less-abundant proteins present. The blood, cerebrospinal fluid, or urine can be from humans or any other animal.

In various embodiments, a sample is prepared for proteomic analysis by substantially removing three or more abundant proteins from blood plasma and/or serum. In one embodiment, chromatographic columns and ‘affinity disks’ configured in series serve to remove, for example, but without limitation, six abundant proteins from serum and/or plasma (e.g., human serum albumin (“HSA”), serotransferrin, immunoglobulin G (“IgG”), orosomucoid, fibrinogen, immunoglobulin A, haptoglobin, alpha-2-macroglobulin, immunoglobulin M, C3 complement, and alpha-1-antitrypsin). These proteins are substantially removed one at a time as the sample traverses each chromatographic element. In certain other embodiments, using this technique, three abundant proteins, four abundant proteins, five abundant proteins, six abundant proteins, or more than six abundant proteins are substantially removed from a sample.

As used herein, a protein is substantially removed when about 95% or greater of the protein is removed, preferably when about 99% or greater of the protein is removed; and, in various embodiments, preferably when the protein to be removed is substantially undetectable in the depleted sample.

In various embodiments, albumin, immunoglobulin G, fibrinogen, and transferrin are removed quantitatively from human plasma using a device we refer to as a “FATIGUE” Cartridge for short, the acronym “FATIGUE” referring to the proteins depleted: F(ibrinogen), A(lbumin), T(ransferrin), and I(mmunoglobulin)G, where the UE is used for phonetic reasons. In one embodiment, a single chromatographic cartridge is packed with materials to remove multiple abundant proteins from plasma or serum in one step. For example, but without limitation, HSA, IgG, fibrinogen, and transferrin can be removed. Four types of supports, each with an affinity to one of the proteins to be removed, are packed within the cartridge in layers. Fewer than four or more than four layers (for example, three, five, or six or more layers) can be used. The supports also can be mixed so that no layers exist.

One aspect of the invention relates to a method for depleting a sample of at least three proteins including the step of contacting a sample with at least one chromatographic medium, where the at least one chromatographic medium is capable of removing albumin, IgG, and a third abundant protein from the sample. This aspect of the invention can have any one or more of the following features. The at least one chromatographic medium can include two or more different chromatographic surfaces for removing two or more different proteins. The two or more chromatographic surfaces can be arranged in series. The sample can be a biological sample. The chromatographic medium can be present in a chromatography column, and the chromatographic column can include particles and/or can include an immunoaffinity chromatography surface. The chromatographic medium can include a chromatography disk. The sample can be moved with a liquid chromatography apparatus.

Another aspect of the invention relates to an apparatus for analyzing a sample of molecules including a liquid chromatography pumping apparatus and at least one chromatographic medium, where the at least one chromatographic medium is capable of removing albumin, IgG, and a third abundant protein from the sample. This aspect of the invention can have any one or more of the following or preceding features. The at least one chromatographic medium can include one or more chromatography columns arranged in series with another chromatography column. The at least one chromatographic medium can include one or more chromatography disks arranged in series with a chromatography column. The apparatus can further include an optical detector and data recorder for said detector. The apparatus can include a computer for controlling sample motion. The third abundant protein can be selected from the group consisting of transferrin, orosomucoid, fibrinogen, IgA, haptoglobin, alpha-2-macroglobulin, immunoglobulin M, C3 complement, and alpha-1-antitrypsin. The method can further include removing a fourth abundant protein. The fourth abundant protein can be selected from the group consisting of transferrin, orosomucoid, fibrinogen, IgA, haptoglobin, alpha-2-macroglobulin, immunoglobulin M, C3 complement, and alpha-1-antitrypsin. The method can further include removing a fifth abundant protein. The fifth abundant protein can be selected from the group consisting of transferrin, orosomucoid, fibrinogen, IgA, immunoglobulin A, haptoglobin, alpha-2-macroglobulin, immunoglobulin M, C3 complement, and alpha-1-antitrypsin. The method can further include removing a sixth abundant protein. The sixth abundant protein can be selected from the group consisting of transferrin, orosomucoid, fibrinogen, IgA, haptoglobin, alpha-2-macroglobulin, immunoglobulin M, C3 complement, and alpha-1-antitrypsin.

Another aspect of the invention relates to a device for substantially removing a component of a sample including, in series, a chromatography column and a chromatography disk.

Another aspect of the invention relates to a device for substantially removing a component of a sample, the device including a cartridge containing at least one chromatographic medium which includes at least a first type and a second type of chromatographic surface. The first type of chromatographic surface can be located in a first layer and the second type of chromatographic surface can be located in a second layer

It should be understood that not all embodiments require removal of albumin and IgG. The invention relates, in various embodiments, to removal of any two or more abundant proteins from a sample. Moreover, in various embodiments, non-abundant proteins can be removed from a sample, rather than abundant proteins, particularly where such non-abundant proteins have a deleterious affect on the ability to analyze a sample or to analyze a component or components therein.

As used herein, the term “chromatography disk” refers to a chromatographic support comprising a monolithic solid phase based on a highly cross-linked porous monolithic polymer with an agglomeration-type or fibrous microstructures. Although the term “disk” is used, those of ordinary skill in the art will recognize that suitable chromatography disks for use in accordance with the invention can have a variety of form factors including, but not limited, cylindrical form factors. Preferred polymeric materials for chromatography disks include, but are not limited to, a poly(glycidylmethacrylate-ethyleneglycol dimethacrylate) or a styrene-divinylbenzene copolymer. In addition, preferred chromatography disks are engineered in such a way that there is essentially no void volume between individual segments of the support matrix, so that the mobile phase is driven through the whole volume of the matrix. Preferred chromatographic disks include those produced by Bia Separation, Inc. Ljubljana, Slovenia Convective Interaction Media® (CIM®) disk technology. Preferred affinity disks include those produced using CIM® disk technology.

As used herein, the term “affinity disk” refers to a chromatography disk that is functionalized for affinity chromatography, for example, by active groups located on the surface of the pores of the support matrix. Examples of suitable affinity chromatography include, but are not limited to, metal affinity chromatography, dye-based affinity chromatography, immunoaffinity chromatography (using, e.g., antibody based affinity), and combinations thereof.

The foregoing and other objects and features of the invention described above will be more fully understood from the following description of various illustrative embodiments, when read together with the accompanying drawings. In the drawings, like reference characters generally refer to the same parts throughout the different views. The drawings are not necessarily to scale, and emphasis instead is generally placed upon illustrating the principles of the invention.

### BRIEF DESCRIPTION OF THE DRAWINGS

FIG. 1 is a schematic diagram of sample flow through an abundant protein removal (“APR”) column train with an in-line C-18 desalting column. The Protein A and anti-HSA columns are packed media, and the CIM® devices are monolithic disks (see inset picture). These disks are secured in a variable length housing which allows for easy addition and/or exchange.

FIG. 2 depicts a 4-12% SDS-NuPAGE™ gel analysis. Sample loads were equal volumes of 8 μg of whole plasma or serum. The lanes are as follows: 1) whole serum, 2) depleted serum, 3) serum proteins bound to anti-HSA, Protein A, anti-transferrin and anti-α-1 antitrypsin columns, 4) serum proteins bound to Protein A column, 5) serum proteins bound to anti-transferrin and anti-α-1-antitrypsin columns, 6) plasma proteins bound to Protein A column, 7) plasma proteins bound to anti-HSA column, 8) plasma proteins bound to anti-transferrin and anti-α-1 antitrypsin columns, 9) plasma proteins bound to anti-orosomucoid and anti-fibrinogen columns.

FIG. 3A depicts a western blot analysis of depleted plasma proteins probed with anti-orosomucoid, anti-α-1-antitrypsin, anti-fibrinogen, and anti-transferrin. The lanes are as follows: 1) depleted plasma, 2) proteins bound to Protein A column, 3) proteins bound to anti-HSA column, 4) proteins bound to anti-transferrin, anti-α-1-antitrypsin, anti-orosomucoid, and anti-fibrinogen columns.

FIG. 3B depicts an anti-IgG and anti-HSA western blot analysis of plasma proteins. The lanes are as follows: 1) depleted plasma—100th run on column, 2) depleted plasma—140th run on column, 3) whole plasma.

FIGS. 4A-4C depict a 2D gel separation of plasma proteins. The first dimension is pH 3-10 non-linear, and the second dimension is 14% SDS-PAGE. The vertical axis of FIGS. 4A-C should read “Molecular Weight, in kDa (kilodaltons)”, and the horizontal axis is “pI” (isoelectric point).

FIG. 5 depicts an SDS-PAGE gel of original and depleted plasma samples. Samples were run on a 12% SDS-PAGE gel and stained with Coomassie Blue. The lanes are as follows: 1) original plasma—20 μg, 2) plasma sample after passage through FATIGUE cartridge—5.8 μg, 3) plasma sample after passage through FATIGUE cartridge—20 μg.

FIG. 6 depicts a western blot analysis of original and depleted samples. Samples were run on a 12% SDS-PAGE gel, blotted onto a PVDF membrane, and IgG was detected using antibody and 1-Step TMB-Blotting reagent (A) or fibrinogen (B) and transferrin (C) using antibodies and ECL Plus™ western blotting detection reagents. The lanes are as follows: 1) original plasma sample—1.4 μg (A) or 0.45 μg (B and C), 2) plasma sample after passage through FATIGUE cartridge—0.41 μg (A) or 0.13 μg (B and C), 3) plasma sample after passage through FATIGUE cartridge—1.4 μg (A) or 0.45 μg (B and C).

### DETAILED DESCRIPTION OF THE INVENTION

Removal of proteins from a sample can be beneficial in order to enhance detection of other components of a sample or to increase sensitivity of assays utilizing the sample. These proteins either can be abundant in the sample or can be present in relatively minor quantities. For example, as described above, a problem in proteomic analysis of complex samples such as blood plasma and serum is the presence of highly abundant proteins which are present at levels higher than those proteins at the low concentration end of the spectrum. The removal of abundant proteins can facilitate the detection of lower concentration proteins. Accordingly, in one aspect, methods and devices according to the invention facilitate the removal of abundant proteins from blood serum or blood plasma. The objective of removing abundant proteins is to facilitate accessing and analyzing one or more less-abundant proteins present. Any sample can be utilized that contains protein, including, but not limited to, blood plasma, blood serum, cerebrospinal fluid, and urine. Such a sample can be from a human or any other animal.

Serum albumin, immunoglobulins, fibrinogen, and transferrin, are non-limiting examples of proteins that may be removed from a sample such as blood plasma and/or serum. These proteins are considered abundant in a sample of blood plasma and/or serum. Serum albumin is the most abundant protein in plasma, representing 40% to 60% of the total protein mass therein. Further, for example, serum derived from plasma in which blood cells are suspended and transported through the body with clotting factors removed, contains 60 to 80 mg of protein per mL, of which, albumin can constitute 35-50 mg/mL. Although serum albumin can serve as a biological marker for identification of plasma oncotic pressure, at such concentrations it represents a problem in searching for new biological markers. Currently, two approaches are typically used to remove albumin from plasma samples: cross-linked agarose gel with covalently attached Cibacron Blue and immunoaffinity chromatography with anti-HSA linked to various supports.

Immunoglobulins are unique in their heterogeneity as their presence usually reflects antigenic stimulation. Based on their structural features, they are divided into the five major classes including immunoglobulin M (IgM), immunoglobulin G (IgG), immunoglobulin A (IgA), immunoglobulin D (IgD) and immunoglobulin E (IgE). Among these, the IgG class represents the second most abundant protein in plasma, representing 8% to 28% of the total protein mass therein. The primary method for quantitative removal (or isolation) of IgG from plasma is the use of affinity chromatography with commercially available bacterial protein G or recombinant Protein A/G attached to agarose gel.

The third most abundant protein in human plasma is fibrinogen. Fibrinogen is a coagulation protein synthesized in liver and is present in plasma at amounts of 3% to 6% of the total plasma protein mass. Two one step methods were developed for isolation of fibrinogen from plasma. The first method is based on the fact that fibrinogen has a strong affinity for fibrin. Accordingly, immobilized fibrin is used to isolate fibrinogen from plasma the second method uses the Gly-Pro-Arg-Pro-Lys peptide immobilized on CDI-activated Fractogel. It has been shown that the N-terminal sequence Gly-Pro-Arg of the α-chain of fibrin is involved in the initiation of the fibrin polymerization by binding to the complementary binding site of an another fibrin(ogen) molecule.

Similar to fibrinogen, transferrin represents 3% to 6% of total plasma protein mass. Transferrin is the primary iron transport protein, and its concentration correlates with the total iron-binding capacity of plasma.

Devices and methods according to the invention can accomplish removal of protein from a sample in any of a variety of ways. Two such ways are described in the examples below. In one manner, in general, a series of at least one column and at least one affinity disk is used to substantially remove various sample components. These columns and disks can be ordered in any of a variety of orders, depending upon the order of component removal desired. Alternatively, a series of two or more columns or a series of two or more affinity disks can be used. In another manner, generally, two or more types of chromatography beads, each having an affinity for a different sample component, can be packed into a single cartridge. Typically, each type of bead is present in a layer separate from the other types of beans. However, the bead types can be intermingled in a single layer.

After the sample from which a protein is substantially removed is collected, the depleted sample can be subjected to a mass spectrographic analysis. Specific examples of protein removal systems according the invention are described in the examples below. These examples are not limiting.

### EXAMPLE 1

### Sample Preparation for Proteomic Analysis by Removal of Abundant Proteins from Blood Plasma and Serum

Described below is a technique which uses multi-column immuno-affinity and on-line reversed phase chromatography to deplete six abundant proteins from human plasma and serum and to desalt the resulting solution. The presence of abundant proteins and subsequent removal are illustrated by the disappearance of densely stained areas that are observed on 2-D electrophoresis gels corresponding to areas where proteins such as HSA and serotransferrin migrate. In addition to HSA and serotransferrin, IgG, orosomucoid, fibrinogen and alpha-1-antitrypsin are quantitatively removed from 50 μL aliquots of serum or plasma. The specificity of the technique is demonstrated, and the benefit is an increase in the dynamic range of protein detection by mass spectroscopy. The device and method used in this experiment are described below.

Anti-HSA and Protein A columns were purchased from Applied Biosystems, Framingham, Mass. Additional antibodies, purchased from DakoCytomation, Carpinteria, Calif., were antigen affinity purified. The affinity disks were produced using Bia Separation, Inc. Ljubljana, Slovenia Convective Interaction Median® (CIM®) disk technology. A CIM® disk monolithic column consists of a CIM® disk in a specially designed housing. The CIM® housing provides low dead volume, excellent sample distribution, simple handling, and can be easily connected to any LC/HPLC or FIA system. The CIM® disk consists of a CIM® matrix and a non-porous, self-sealing fitting ring that ensures only axial flow through the disk and prevents any sample and mobile phase leakage or by-pass. This ensures that all of the mobile phase is driven through the entire volume of the matrix. Additionally, the CIM® matrix has a well-defined pore-size distribution providing excellent separations at low back pressure. Since all CIM® disks have the same dimensions, they fit in the same housing enabling assembly of various CIM® disk monolithic columns by simply changing the chemistry while the housing is still attached to the HPLC/FIA system. This advantageous feature allows the construction of an analytical column that is any desired configuration. Resolution can be increased, for example, by placing up to 4 disks of the same chemistry into one housing. CIM® disk monolithic supports provide the ability to assemble a CIM® Conjoint Liquid Chromatography (CLC) disk monolithic column. CLC columns allow single step protein separations and purifications by placing up to 4 CIM® disks with different active groups into one housing. In addition, CIM® disk monolithic supports have a well defined distribution of flow-through pores which permit low back pressure even at high mobile phase flow rates, fast mass transfer (based on convection), and a large surface area creating high binding capacities.

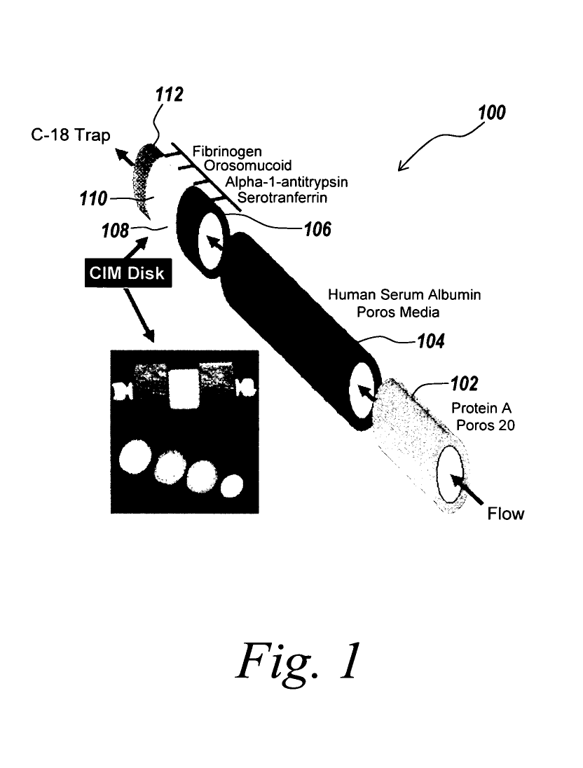

The CIM® disks were modified with Protein A to enable quick immobilization of specific antibodies. These antibodies were then crosslinked to the Protein A using dimethyl pimelimidate. Ultimately, referring to FIG. 1, an assembly was produced 100 that included a Protein A column 102 which was followed in serial fluidic communication by an anti-HSA column 104 which was followed in serial fluidic combination by CIM® disks containing, in order, anti-serotransferrin 106, anti-alpha-1-antitrypsin 108, anti-orosomucoid 110, and anti-fibrinogen antibodies 112.

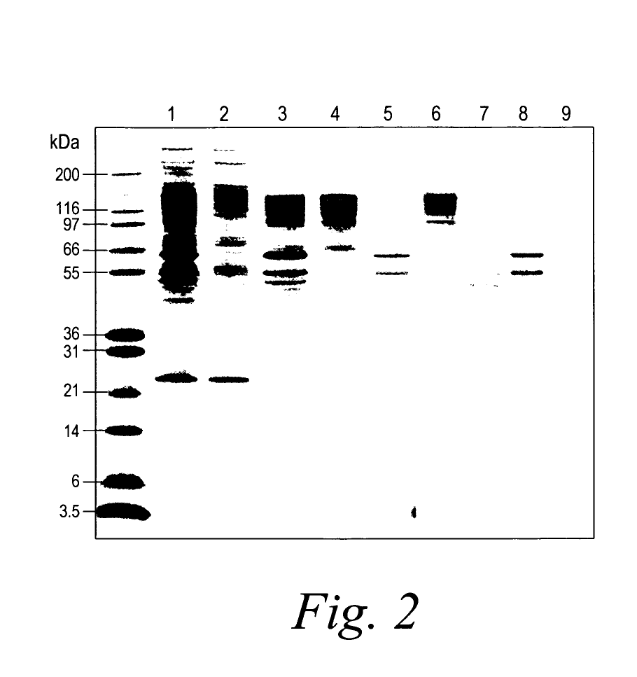

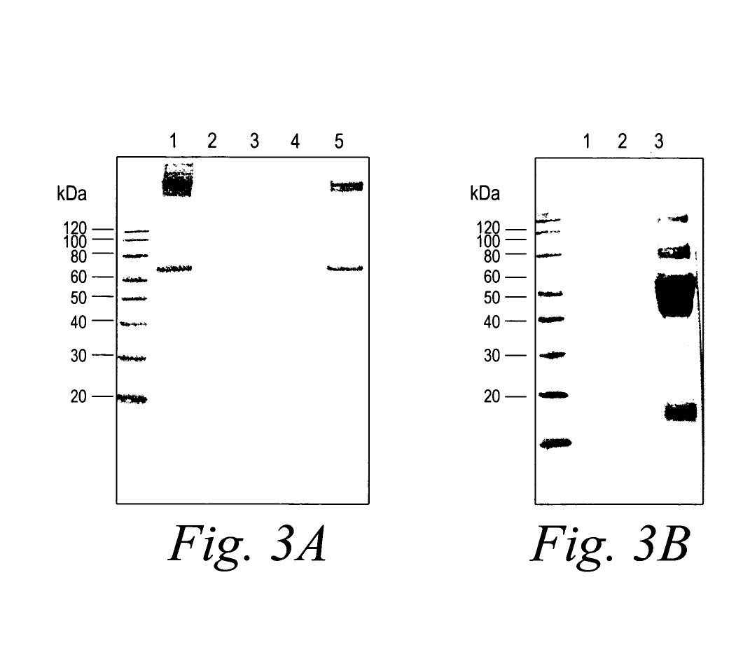

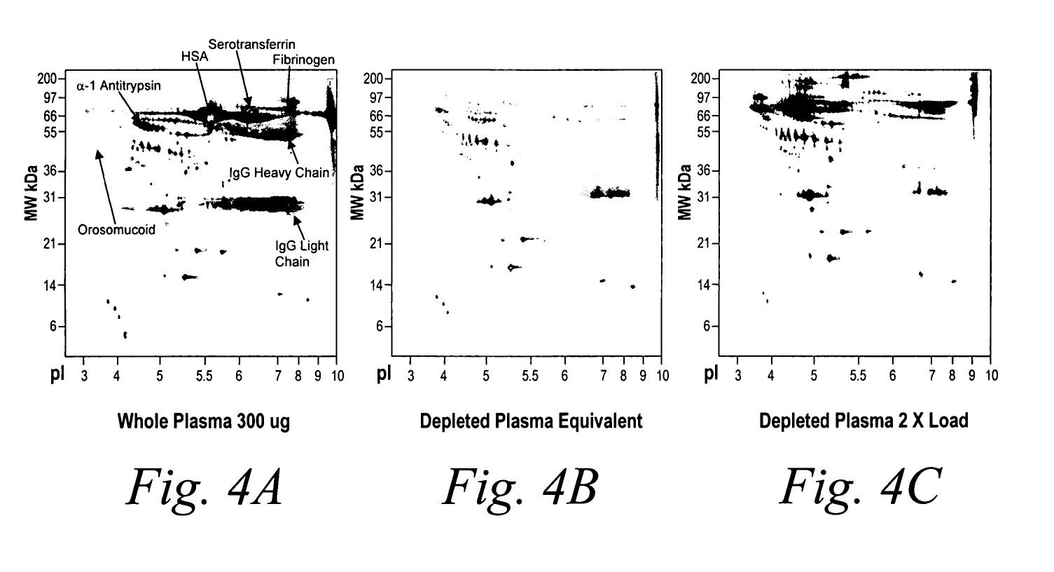

Plasma or serum samples (50 μL) were injected on to the series of APR columns and the flow-through peak was trapped on a C-18 reversed phase column. The C-18 column was then eluted with a step gradient to 100% acetonitrile. This fraction constitutes the depleted sample. The individual APR columns were then eluted with 200 mM glycine, pH 2.5, and were collected on an equal volume basis. Fractions were neutralized with NaOH. Affinity captured and C-18 desalted fractions were analyzed by 1D (FIGS. 2, 3A and 3B) and 2D SDS-PAGE gels (FIGS. 4A-4C) either with silver staining or with western blotting. To carry out these procedures, 1-D NuPAGE™ gels, a SilverXpress™ Silver Staining Kit, and western blotting material were purchased from Invitrogen, Carlsbad, Calif. The 2D analysis used a 3-10 non-linear pI gradient, IPG strip from Amersham Biosciences, Piscataway, N.J. Samples were 300 μg of whole plasma and equivalent APR depleted plasma and were cup loaded onto the first dimension, 3500 V for 15 hr. Second dimension gels were 14% acrylamide. Western blots were probed using antigen specific rabbit IgG fraction. Secondary anti-rabbit IgG horse radish peroxidase conjugate was used to visualize by chemiluminescence or colorimetric methods.

As a result of the construction of the device and subsequent testing, immobilized antibody columns and affinity disks were used to remove six of the most abundant proteins from 50 μl loads of plasma and serum with little non-specific binding of proteins (FIGS. 2, 3A, and 3B). The order of the columns (FIG. 1) is important in the removal scheme, because the Protein A column serves as a guard column as well as for IgG removal. The anti-HSA column removes this abundant protein (HSA) prior to the CIM® disk devices. The C-18 reversed phase column serves as both a concentrating and desalting device. The use of CIM® disks (FIG. 1) allowed for multiple protein removal while minimizing the peak band broadening and volume increase of the sample. There was no evidence of nonspecific binding among the various column media (FIGS. 2, 3A, and 3B). The anti-HSA and Protein A columns continued to remove over 98% of HSA and IgG after as many as 140 runs (FIG. 3B). Removal of the six abundant proteins permits the separation of proteins which are typically obscured. These observed improvements in 1D and 2D gel electrophoresis increase the ability to isolate and identify low abundant proteins (FIGS. 2 and 4A-C). This APR method also leads to improved tryptic digest due to the decreased sample complexity and removal of the inhibitory alpha-1-antitrypsin. Mass spectrometric analysis of plasma and serum samples after abundant protein removal show few or no peptides from the removed proteins and has allowed for identification of lower abundance proteins. It is contemplated that additional abundant proteins will be targeted in order to further optimize the dynamic range of protein concentrations in plasma. It is also contemplated that samples other than blood plasma and/or serum can be used, including, but not limited to, cerebrospinal fluid and urine.

### EXAMPLE 2

### Quantitative Removal of Albumin, Immunoglobulin G, Fibrinogen, and Transferrin from Human Plasma Using the FATIGUE Cartridge

This example is focused on quantitative removal of the four most abundant proteins from human plasma. Serum albumin, IgG, fibrinogen, and transferrin were quantitatively removed from plasma using one step affinity chromatography. This process uses a cartridge filled with four types of supports, each designed to capture one of the proteins listed above.

Materials

Blue Sepharose™ 6 Fast Flow (Cibacron Blue F3G-A, covalently bound ligand, which is coupled with highly cross-linked agarose), HRP-linked anti-rabbit IgG secondary antibody, and ECL Plus™ western blotting detection reagents were purchased from Amersham; UltraLink Immobilized Protein A/G (Protein A/G is a genetically engineered protein that combines the IgG binding profiles of both Protein A and Protein G) and 1-Step TMB-Blotting (a system where a compound produces a calorimetric signal) were purchased from Pierce Biotechnolgy, Rockland, Ill.; Affi-Prep 10 (a support that consists of polymeric macroporous (1,000 Å) 45 μm beads with USP Grade polymyxin B covalently attached), Affi-Gel 102 (Supports for use with EDAC coupling reagent immobilize ligands containing primary or terminal carboxyl groups), and EDAC coupling reagent (1-ethyl-3-(3-dimethylaminopropyl)carbodiimide) were purchased from Bio-Rad, Hercules, Calif.; and all primary antibodies used in this work were purchased from DAKO.

Preparation of Gly-Pro-Arg-Pro-Affi-Gel 102 Conjugate

Peptide Gly-Pro-Arg-Pro was synthesized and was released from the cartridge without unblocking amino and carboxyl groups. Lyophilized peptide (approximately 50 μmoles) was resolved in 3.0 ml of 50% ethanol and 50 mM sodium acetate, pH 6.0. 2 ml Affi-Gel 102 was diluted with 2 ml distilled water, and 1.5 ml dissolved peptide was added. After the pH was adjusted to approximately 4.8 with 1N HCl, 10 mg EDAC coupling reagent was added, and the pH was readjusted to approximately 4.8. The reaction proceeded overnight at room temperature with continued gentle stirring. Unbound peptide was washed out with distilled water. For deprotection of the immobilized peptide, the gel was resuspended in 3 ml TFA and gently mixed for 2 hrs. TFA was washed out with distilled water and PBS-8 buffer (20 mM phosphate buffer, pH 8.0; 150 mM NaCl). A Gly-Pro-Arg-Pro-Affi-Gel 102 conjugate resulted.

Immobilization of Anti-Transferrin Antibody on Affi-Prep 10 Support

1 ml of Anti-human transferrin polyclonal antibody (14 mg/ml) was dialyzed for 2 hrs against 50 mM sodium acetate, pH 4.5, using a Slide-A-Lyzer Dialysis Cassette (Pierce), diluted to 4.5 ml with the same buffer, and gently mixed with 1 ml Affi-Prep 10 support previously washed with 10 mM sodium acetate, pH 4.5. The coupling reaction was carried out for 2 hrs at room temperature, and unbound antibody was removed by washing the support with 100 mM Hepes buffer, pH 7.4, and PBS-8. Mobilization of anti-transferrin antibody on an Affi-Prep 10 Support resulted.

Packing of FATIGUE Cartridge

A disposable polypropylene column (1-5 ml bed volumes; Pierce Biotechnology, Rockland, Ill.) was packed in this order of supports: 0.3 ml anti-transferrin-Affi Gel 100 support, 0.4 ml Gly-Pro-Arg-Pro-Affi-Gel 102 support, 0.5 ml UltraLink Immobilized Protein A/G support, and 1 ml Blue Sepharose™ 6 Fast Flow support. Accordingly, the FATIGUE cartridge had four layers containing these four types of supports. A frit was placed on the top of the packed supports, and the column was equilibrated with 20 ml PBS-8 buffer.

Removal of Serum Albumin, IgG, Fibrinogen and Transferrin from Plasma Sample

In a typical experiment, 100 μl of plasma was loaded into the FATIGUE cartridge followed by a wash with 0.5 ml PBS-8 buffer. Unbound proteins were then eluted from the cartridge with 8 ml PBS-8/300 (20 mM phosphate buffer, pH 8.0; 300 mM NaCl), and 1 ml fractions were collected. Protein concentration in individual fractions was determined by A₂₈₀ (a spectrophotometric technique), and fractions containing proteins were pooled and further tested for the presence of serum albumin, IgG, fibrinogen, and transferrin by SDS-PAGE and Western blots. Proteins bound to the column were desorbed with 10 ml PBS-8/300 containing 6 M urea and re-equilibrated with 10 ml of PBS-8 for further use.

Results and Discussion

6.5 mg of plasma proteins were loaded into the FATIGUE cartridge, and elution of unbound proteins was carried out as described above. Fractions containing proteins (the first four fractions) were eluted and pooled (3.5 ml). The concentration was determined by A₂₈₀ at 0.54 mg/ml which represents 1.89 mg of unbound plasma proteins (29% of original plasma protein content). Individual or pooled fractions were further investigated for the presence of serum albumin, immunoglobulin G, fibrinogen, and transferrin by SDS-PAGE and Coomassie Blue staining or Western blot analysis.

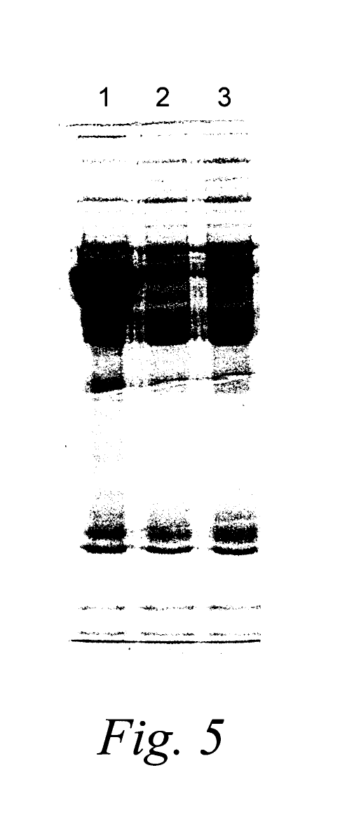

For removal of albumin, the Blue Sepharose™ 6 Fast Flow support in which Cibacron Blue is coupled with highly cross-linked agarose was used. The original plasma sample (lane 1), pooled fractions representing the same volume of the original plasma sample as was loaded in lane 1 (lane 2), and pooled fractions representing the same protein amount as in the original plasma sample as was loaded in lane 1 (lane 3) were run on a 12% SDS-PAGE gel and Coomassie stained (FIG. 5). As seen in FIG. 5, almost all of that albumin was adsorbed to the Cibacron Blue in the FATIGUE cartridge. Western blot analysis shows that serum albumin is present in the depleted sample in an amount less than 1%.

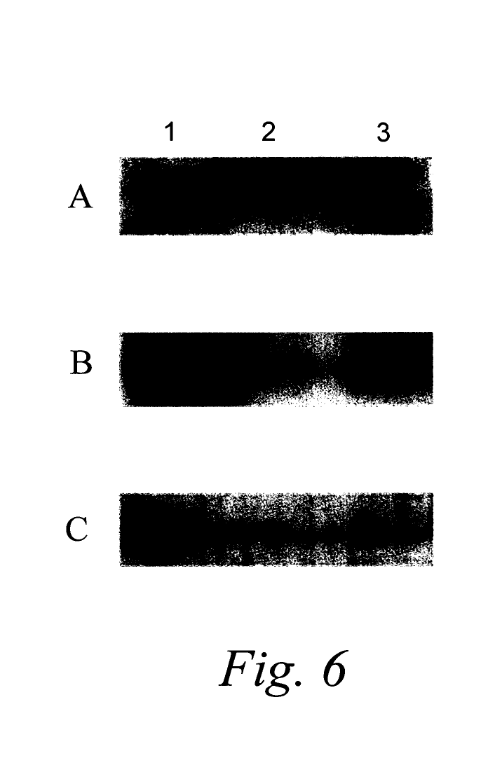

Immunoglobulin G was removed from the plasma sample using UltraLink Immobilized Protein A/G from Pierce. The IgG concentrations in the original and depleted samples were determined by Western blot analysis (FIG. 6A). 7.5 μl of 350 times diluted original plasma sample (1.4 μg; lane 1), 7.5 μl of 10 times diluted depleted sample representing the same volume as was loaded in lane 1 (0.41 μg; lane 2), or 26 μl of 10 times diluted depleted sample representing the same protein amount as was loaded in lane 1 (1.4 μg; lane 3) were run on a 12% SDS-PAGE gel and were blotted onto a PVDF membrane. The presence of immunoglobulin G was determined using anti-Ig G antibody and 1-Step TMB-Blotting reagent. As can be seen from FIG. 6A, the only detectable IgG is present in the original plasma sample, while the depleted sample is essentially free of this protein.

In order to remove fibrinogen, Gly-Pro-Arg-Pro-Affi-Gel 102 Conjugate was used. This is a modification of the method described by Travis, J., and Pannell, R. in “Selective removal of albumin from plasma by affinity chromatography,” Clin Chim Acta, 1973, 49: 49-52, the entire contents of which is hereby incorporated by references. In the present example, only the tetrapetide linked by the Pro carboxyl group to a 6-atom N-terminal hydrophilic arm was used. Transferrin was removed using anti-human transferrin coupled to Affi-Prep 10 gel. The concentrations of fibrinogen (FIG. 6B) and transferrin (FIG. 6C) in the original plasma and the depleted samples were determined by Western blot (ECL Plus™ western blotting). For both fibrinogen and transferrin, 10 μl of 1000 times diluted original plasma (0.45 μg; lane 1), 10 μl of 30 times diluted depleted sample representing the same volume as was loaded in lane 1 (0.13 μg; lane 2), or 34 μl of 30 times diluted depleted sample representing the same protein amount as was loaded in lane 1 (0.45 μg; lane 3) were run on a 10% SDS-PAGE gel, and blotted onto a PVDF membrane. The presence of fibrinogen or transferrin was determined using anti-fibrinogen or anti-transferrin antibody and ECL Plus™ reagent. The concentrations of fibrinogen or transferrin dropped to less than 5% or 20% in the depleted sample, respectively, as determined by densitometry.

As seen from data above, the FATIGUE cartridge is efficient in removing the main protein components from plasma and facilitates identifying and characterizing proteins which are present in the plasma in small quantities. It may be that another technique can be used to remove albumin in the FATIGUE cartridge because it is possible that Cibacron Blue also can bind to other proteins (particularly lipoproteins). For example, anti-human albumin antibody cross-linked to the various supports could be used. However, it was shown by quantitative radial immunodiffusion that α₁-antitrypsin, α₂-macroglobulin, transferrin, haptoglobin, ceruloplasmin, and orosomucoid were recovered quantitatively. The concentration of α₁-antitrypsin in the original and depleted plasma samples was measured. A western blot analysis indicated that α₁-antitrypsin is recovered quantitatively without any significant losses. Additionally, it was found that all bound proteins in the FATIGUE cartridge were easily eluted with 6 M urea and further analyzed for the protein contents.

The claims should not be read as limited to the described order or elements unless stated to that effect. While the invention has been particularly shown and described with reference to specific illustrative embodiments, it should be understood that various changes in form and detail may be made without departing from the spirit and scope of the invention as defined by the appended claims. By way of example, any of the disclosed features may be combined with any of the other disclosed features to substantially remove proteins in accordance with the invention. Therefore, all embodiments that come within the scope and spirit of the following claims and equivalents thereto are claimed as the invention.

1. A method for depleting a sample of at least three proteins comprising the step of contacting a sample with at least one chromatographic medium, the at least one chromatographic medium being capable of removing albumin, IgG, and a third abundant protein from the sample.

2. The method of claim 1 wherein the at least one chromatographic medium comprises two or more different chromatographic surfaces for removing two or more different proteins.

3. The method of claim 1 wherein the sample is one or more of blood plasma, blood serum, cerebrospinal fluid, and urine.

4. The method of claim 1 wherein the chromatographic medium is present in a chromatography column.

5. The method of claim 4 wherein the chromatographic column comprises particles.

6. The method of claim 4 wherein the chromatographic column comprises an immunoaffinity chromatography surface.

7. The method of claim 1 wherein the chromatographic medium, comprises a chromatography disk.

8. The method of claim 1 wherein the sample is moved with a liquid chromatography apparatus.

9. An apparatus for analyzing a sample of molecules comprising a liquid chromatography pumping apparatus and at least one chromatographic medium, the at least one chromatographic medium being capable of removing albumin, IgG, and a third abundant protein from the sample.

10. The apparatus of claim 9 wherein the at least one chromatographic medium comprises one or more chromatography columns arranged in series with another chromatography column.

11. The apparatus of claim 9 wherein the at least one chromatographic medium comprises one or more chromatography disks arranged in series with a chromatography column.

12. The method of claim 1 wherein the third abundant protein is selected from the group consisting of transferrin, orosomucoid, fibrinogen, immunoglobulin A, haptoglobin, alpha-2-macroglobulin, immunoglobulin M, C3 complement, and alpha-1-antitrypsin.

13. The method of claim 1 further comprising removing a fourth abundant protein.

14. The method of claim 13 wherein the fourth abundant protein is selected from the group consisting of transferrin, orosomucoid, fibrinogen, immunoglobulin A, haptoglobin, alpha-2-macroglobulin, immunoglobulin M, C3 complement, and alpha-1-antitrypsin.

15. The method of claim 14 further comprising removing a fifth abundant protein.

16. The method of claim 15 wherein the fifth abundant protein is selected from the group consisting of transferrin, orosomucoid, fibrinogen, immunoglobulin A, haptoglobin, alpha-2-macroglobulin, immunoglobulin M, C3 complement, and alpha-1-antitrypsin.

17. The method of claim 16 further comprising removing a sixth abundant protein.

18. The method of claim 17 wherein the sixth abundant protein is selected from the group consisting of transferrin, orosomucoid, fibrinogen, immunoglobulin A, haptoglobin, alpha-2-macroglobulin, immunoglobulin M, C3 complement, and alpha-1-antitrypsin.

19. A device for substantially removing a protein from a sample comprising, in serial fluidic communication, a chromatography column and a chromatography disk.

20. A device for depleting a sample of at least three proteins comprising:
an immunoaffinity chromatography surface for each protein to be depleted from the sample where the chromatographic surfaces are arranged in serial fluidic communication as a single chromatographic column.

21. The device of claim 20, wherein the proteins to be depleted from the sample comprise albumin, transferrin and immunoglobulin G.

22. The device of claim 21, wherein the proteins to be depleted from the sample further comprise haptoglobin, alpha-1-antitrypsin and immunoglobulin A.

23. A device for depleting a sample of at least three proteins comprising:
a first chromatography column functionalized to substantially remove albumin from the sample;
a second chromatography column in serial fluidic communication with the first chromatography column and functionalized to substantially remove immunoglobulin G from the sample; and
a first chromatography disk in serial fluidic communication with the second chromatography column and functionalized to substantially remove a third protein from the sample.

24. The device of claim 23, wherein the third protein is selected from the group consisting of transferrin, orosomucoid, fibrinogen, immunoglobulin A, haptoglobin, alpha-2-macroglobulin, immunoglobulin M, C3 complement, and alpha-1-antitrypsin.

25. The device of claim 23, further comprising a second chromatography disk in serial fluidic communication with the first chromatography disk and functionalized to substantially remove a fourth protein from the sample.

26. The device of claim 25, wherein the fourth protein is selected from the group consisting of transferrin, orosomucoid, fibrinogen, immunoglobulin A, haptoglobin, alpha-2-macroglobulin, immunoglobulin M, C3 complement, and alpha-1-antitrypsin.

27. The device of claim 25, further comprising a third chromatography disk in serial fluidic communication with the second chromatography disk and functionalized to substantially remove a fifth protein from the sample.

28. The device of claim 27, wherein the fifth protein is selected from the group consisting of transferrin, orosomucoid, fibrinogen, immunoglobulin A, haptoglobin, alpha-2-macroglobulin, immunoglobulin M, C3 complement, and alpha-1-antitrypsin.

29. The device of claim 27, further comprising a fourth chromatography disk in serial fluidic communication with the third chromatography disk and functionalized to substantially remove a sixth protein from the sample.

30. The device of claim 29, wherein the sixth protein is selected from the group consisting of transferrin, orosomucoid, fibrinogen, immunoglobulin A, haptoglobin, alpha-2-macroglobulin, immunoglobulin M, C3 complement, and alpha-1-antitrypsin.
